(19) United States
(12) Patent Application Publication
(10) Pub. No.: US 2012/0245534 A1
(43) Pub. Date: Sep. 27, 2012

(54) Drug Delivery Electrode with Temporary Fill Tube

(75) Inventor: Claude Jolly, Innsbruck (AT)

(73) Assignee: MED-EL ELEKTROMEDIZINISCHE GERAETE GMBH, Innsbruck (AT)

(21) Appl. No.: 13/424,465

(22) Filed: Mar. 20, 2012

Related U.S. Application Data
(60) Provisional application No. 61/467,474, filed on Mar. 25, 2011.

(51) Int. Cl. A61M 31/00; A61N 1/05

(52) U.S. Cl. 604/257

(57) ABSTRACT

An ear implant electrode device is described. An implantable electrode array has an outer surface with electrode contacts for electrically stimulating adjacent neural tissue. There is a fluid delivery channel within the electrode array for delivering a therapeutic fluid through one or more fluid delivery ports on the outer surface. A temporary fill tube is connected to the fluid delivery channel for providing the therapeutic fluid when the electrode array is partially inserted into a patient neural tissue without being functional after the electrode array is fully inserted. At least a portion of the fill tube is adapted for removal after providing the therapeutic fluid to have the fluid delivery channel and any remaining portion of the fill tube completely enclosed within the neural tissue after the electrode array has been fully inserted so as to leave no exposed fluid opening outside the neural tissue.

This application claims priority from U.S. Provisional Patent 61/467,474, filed Mar. 25, 2011, which is incorporated herein by reference.

### TECHNICAL FIELD

The present invention relates to an implantable electrode for auditory prostheses.

### BACKGROUND ART

A normal ear transmits sounds as shown in FIG. 1 through the outer ear 101 to the tympanic membrane (eardrum) 102, which moves the bones of the middle ear 103, which in turn vibrate the oval window and round window openings of the cochlea 104. The cochlea 104 is a long narrow duct wound spirally about its axis for approximately two and a half turns. The cochlea 104 includes an upper channel known as the scala vestibuli and a lower channel known as the scala tympani, which are connected by the cochlear duct. The scala tympani forms an upright spiraling cone with a center called the modiolar where the spiral ganglion cells of the acoustic nerve 113 reside. In response to received sounds transmitted by the middle ear 103, the fluid filled cochlea 104 functions as a transducer to generate electric pulses that are transmitted to the cochlear nerve 113, and ultimately to the brain.

Hearing is impaired when there are problems in the ability to transduce external sounds into meaningful action potentials along the neural substrate of the cochlea 104. In some cases, hearing impairment can be addressed by an auditory prosthesis system such as a cochlear implant that electrically stimulates auditory nerve tissue with small currents delivered by multiple electrode contacts distributed along an implant electrode. FIG. 1 shows some components of a typical cochlear implant system where an external microphone provides an audio signal input to an external signal processing stage 111 which implements one of various known signal processing schemes. The processed signal is converted by the external signal processing stage 111 into a digital data format, such as a sequence of data frames, for transmission into a receiver processor in an implant housing 108. Besides extracting the audio information, the receiver processor in the implant housing 108 may perform additional signal processing such as error correction, pulse formation, etc., and produces a stimulation pattern (based on the extracted audio information) that is sent through an electrode lead 109 to an implanted electrode array 112 which penetrates into the cochlea 104 through a surgical opening called a cochleostomy. Typically, this electrode array 112 includes multiple electrode contacts 110 on its surface that deliver the stimulation signals to adjacent neural tissue of the cochlea 104 which the brain of the patient interprets as sound. The individual electrode contacts 110 may be activated sequentially, or simultaneously in one or more contact groups.

There are many inner ear disorders which can lead to some degree of hearing loss. Among these are sudden hearing loss, noise induced hearing loss, progressive hearing loss, aminoglycoside induced hearing loss, presbiyacusis etc., autoimmune inner ear disorder, and infections (bacterial, viral, fungal). Many of the diseases that lead to partial or total hearing loss could utilize a therapeutic pharmaceutical treatment to reach some tissue or cells within the inner ear, for example, to arrest or reverse the hearing loss and improve hearing. Examples of therapeutic pharmaceutical molecules include without limitation cortico-steroids, peptides, and other proteins.

But there are relatively few ways to deliver therapeutic drugs to the inner ear. Typical clinical practice involves either oral, veinous, or arterial drug delivery. Topical drug delivery to treat the inner ear is limited to deposition of the drug at the round window and relying on diffusion of the drug through the round window to reach targeted cells. This may be accomplished by flooding the middle ear cavity with the drug in liquid form, or by applying a soaked sponge at or near the round window, for example, through an opening in the tympanic membrane. But a diffusion process through the round window is not particularly predictable or reliable. The permeability of the round window varies greatly between patients and by other criteria such as time of day, physical conditions, application methods, drug used. Thus, the amount of drug that reaches the inner ear through such delivery methods may vary anywhere between zero and toxically too much.

U.S. Patent Publication 20090259267 describes an implantable electrode with an enlarged fluid storage reservoir that stores a treatment volume of therapeutic fluid for an extended therapeutic treatment period. One or more fluid delivery ports connect the fluid storage reservoir to the outer surface of the electrode for delivering the therapeutic fluid from the fluid storage reservoir to the outer surface.

### SUMMARY

Embodiments of the present invention are directed to an ear implant electrode device. An implantable electrode array has an outer surface with electrode contacts for electrically stimulating adjacent neural tissue. There is a fluid delivery channel within the electrode array for delivering a therapeutic fluid through one or more fluid delivery ports on the outer surface. A temporary fill tube is connected to the fluid delivery channel for providing the therapeutic fluid when the electrode array is partially inserted into patient neural tissue without being functional after the electrode array is fully inserted. At least a portion of the fill tube is adapted for removal after providing the therapeutic fluid to have the fluid delivery channel and any remaining portion of the fill tube completely enclosed within the neural tissue after the electrode array has been fully inserted so as to leave no exposed fluid opening outside the neural tissue.

The fill tube may have a free end that extends into a posterior tympanotomy or even a mastoidectomy when the electrode array is partially inserted. The at least a portion of the fill tube may be adapted for removal by cutting, for example, by cutting the fill tube flush with the outer surface of the electrode array.

Embodiments of the present invention also include a method of delivering a therapeutic fluid with an ear implant electrode. An electrode array is partially inserted into patient neural tissue, which array includes an outer surface including a plurality of electrode contacts for electrically stimulating adjacent neural tissue, a fluid delivery channel within the electrode array for delivering a therapeutic fluid through at least one fluid delivery port on the outer surface, and a temporary fill tube connected to the fluid delivery channel. The therapeutic fluid is provided to the fluid delivery channel via the fill tube to deliver the therapeutic fluid through the at least one fluid delivery port to the outer surface of electrode array. At least a portion of the fill tube is removed and the insertion of the electrode array into the patient neural tissue is completed so that the fluid delivery channel and any remaining portion of the fill tube are completely enclosed within the neural tissue so as to leave no exposed fluid opening outside the neural tissue.

The fill tube may have a free end that extends into a posterior tympanotomy or even a mastoidectomy when the electrode array is partially inserted. And removing the at least a portion of the fill tube may include cutting the fill tube, for example, cutting the fill tube flush with the outer surface of the electrode array.

The ear implant system may include a cochlear implant system, an auditory brainstem implant system, or a vestibular implant system. And embodiments of the present invention also include an ear implant system having an electrode arrangement according to any of the above.

### BRIEF DESCRIPTION OF THE DRAWINGS

FIG. 1 shows anatomical structures of a human ear and some components of a typical cochlear implant system.

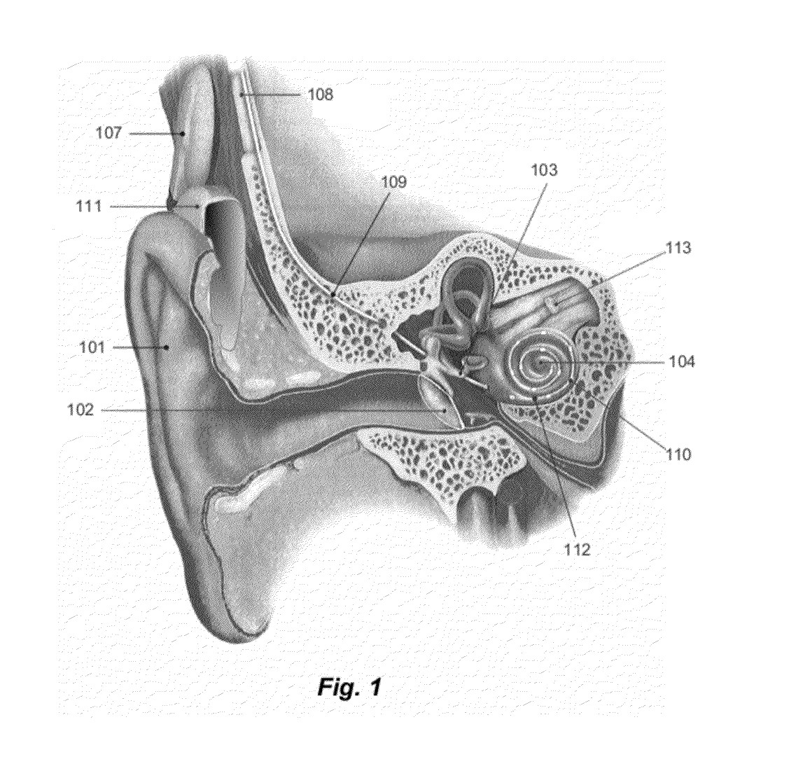

FIG. 2 shows structural details of an implantable cochlear electrode having a temporary fill tube according to an embodiment of the present invention.

FIG. 3 shows a cochlear implant system having an implantable electrode with a temporary fill tube according to an embodiment of the present invention.

FIG. 4 shows various logical steps in implanting a cochlear electrode using a temporary fill tube according to an embodiment of the present invention.

### DETAILED DESCRIPTION

It would be useful to be able to safely deliver therapeutic drugs to the cochlea during implantation of cochlear electrodes. For example, corticosteroid could be useful for the treatment of cochlear inflammation after opening the scala and inserting the electrode. It would also be beneficial to bring other type of therapeutic drugs to the cochlea for acting on the spiral ganglion cells and the cochlear neurites. Some peptides in the form of nerve growth factors also could be useful. With any drug delivery to the inner ear, it is important to bring a precise and controlled dose into the scala at specific locations other that the very base of the cochlea.

In the past, this problem has been addressed by injecting a small amount of drug with a needle at the entrance point of the electrode into the cochlea during implantation. Alternatively, the electrode has been dipped in a drug solution in the hope that a coating of the drug would stay on the electrode during insertion. In another approach, the drug has been placed at the round window of the cochlea before electrode insertion in the hope that some diffusion would occur before, during, or after surgery. Other methods proposed have been a drug delivery reservoir connected to a port and septum for filling or refilling, and to load crystal-form drugs in the silicone carrier material of the electrode and rely on diffusion out of the silicone.

Embodiments of the present invention are directed to an improved ear implant electrode device. This novel approach uses a temporary fill tube that is used after the electrode has been partially inserted to deliver therapeutic drugs to the inner ear. After delivery of the drugs, the fill tube is removed and insertion of the electrode is completed. This ensures that after the implantation surgery all the fluid delivery structures are completely enclosed within the cochlea so as to leave no fluid opening outside the cochlea. This ensures that any future infection in the middle ear does not spread through the implanted electrode into the inner ear, a potentially deadly complication that could otherwise arise.

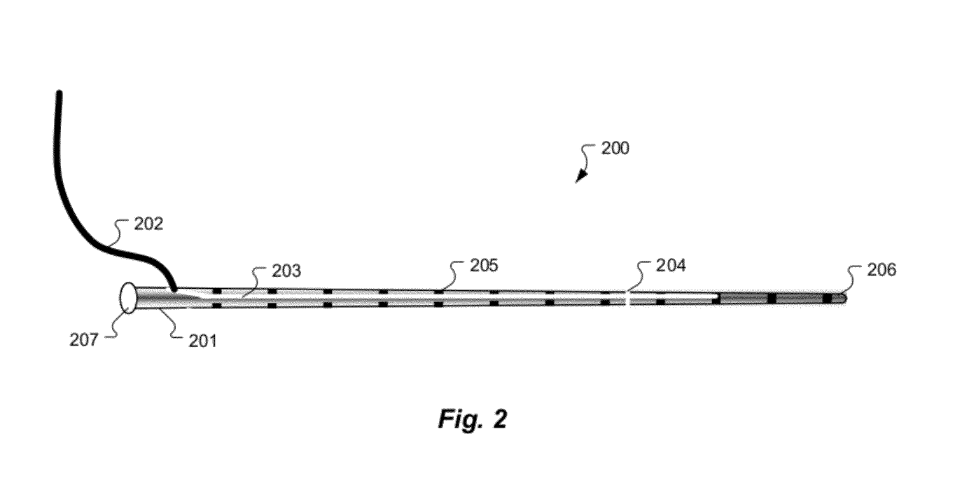

FIG. 2 shows structural details of an implantable ear implant electrode arrangement for a cochlear implant system, an auditory brainstem implant system, or a vestibular implant system. The ear implant arrangement 200 has an implantable electrode array 201 with electrode contacts 205 on its outer surface for electrically stimulating cochlear neural tissue after the device has been inserted in the cochlea. Within the electrode array 201 is a fluid delivery channel 203 for delivering a therapeutic fluid through one or more fluid delivery ports 204 on the outer surface of the array.

A temporary fill tube 202 is connected to the fluid delivery channel 203 for providing the therapeutic fluid when the electrode array 201 is partially inserted into a patient cochlea. The free end of the fill tube 202 may extend back into a posterior tympanotomy or even a mastoidectomy when the electrode array 201 is partially inserted during implantation surgery. At least a portion of the fill tube 202 is adapted for removal after providing the therapeutic fluid so that the fluid delivery channel 203 and any remaining portion of the fill tube 202 remain completely enclosed within the cochlea after the electrode array 201 has been fully inserted so as to leave no fluid opening outside the cochlea. For example, a portion of the fill tube 202 may removable by cutting the fill tube 202 flush with the outer surface of the basal end 207 of the electrode array 201.

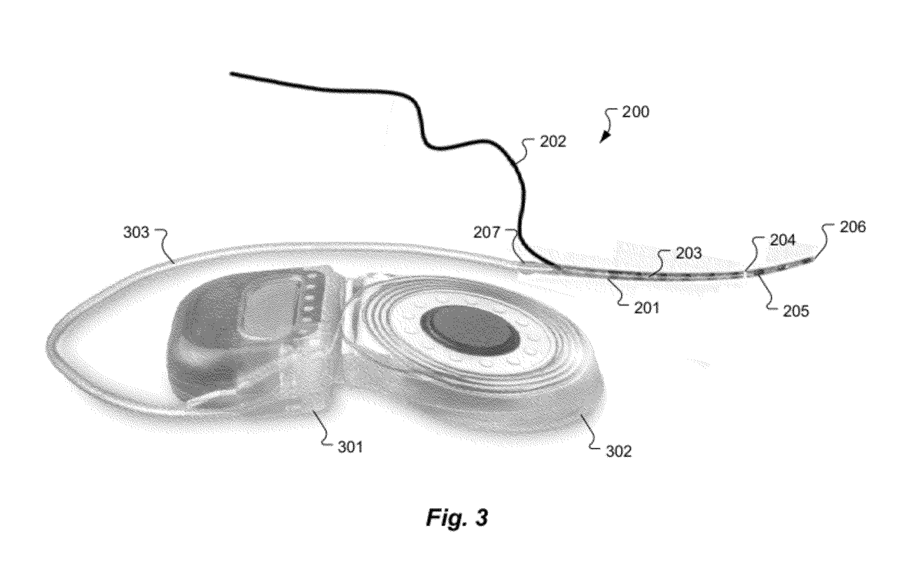

FIG. 3 shows a cochlear implant system having an implantable electrode arrangement 200 having a temporary fill tube 202. That is, FIG. 3 shows how the electrode array 201 extends back as an electrode lead 303 that connects to the housing of an implantable stimulator 301 coupled to an implantable receiver coil 302. The receiver coil 302 receives an externally transmitted implant communications signal which the stimulator 301 converts into the electrical stimulation signals for the electrode contacts 205 of the electrode array 201.

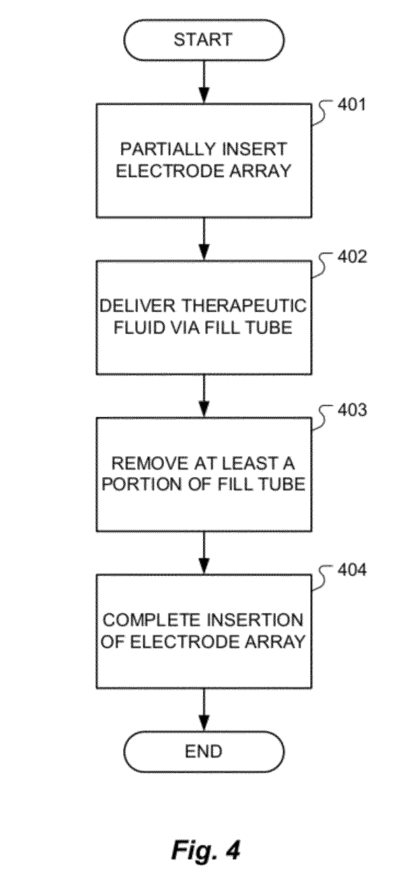

During surgical implantation, the electrode array 201 is partially inserted into the patient cochlea, step 401 in FIG. 4. The therapeutic fluid is provided to the fluid delivery channel 203 via the fill tube 202 to deliver the therapeutic fluid through one or more fluid delivery ports 204 to the outer surface of electrode array 201, step 402. Then at least a portion of the fill tube 202 is removed, step 403, for example, by cutting the fill tube 202 flush with the outer surface of the basal end 207 of the electrode array 201. Once the fill tube 202 has been cut, the insertion of the electrode array 201 into the patient cochlea can be completed, step 404, so that the fluid delivery channel 203 and any remaining portion of the fill tube 202 are completely enclosed within the cochlea so as to leave no fluid opening outside the cochlea.

Although various exemplary embodiments of the invention have been disclosed, it should be apparent to those skilled in the art that various changes and modifications can be made which will achieve some of the advantages of the invention without departing from the true scope of the invention.

What is claimed is:

1. An ear implant electrode device comprising:
an implantable electrode array having an outer surface including a plurality of electrode contacts for electrically stimulating adjacent neural tissue;
a fluid delivery channel within the electrode array for delivering a therapeutic fluid through at least one fluid delivery port on the outer surface; and
a temporary fill tube connected to the fluid delivery channel for providing the therapeutic fluid when the electrode array is partially inserted into patient neural tissue without being functional after the electrode array is fully inserted;
wherein at least a portion of the fill tube is adapted for removal after providing the therapeutic fluid to have the fluid delivery channel and any remaining portion of the fill tube completely enclosed within the neural tissue after the electrode array has been fully inserted so as to leave no exposed fluid opening outside the neural tissue.

2. An ear implant electrode device according to claim 1, wherein the fill tube includes a free end that extends into a posterior tympanotomy when the electrode array is partially inserted.

3. An ear implant electrode device according to claim 1, wherein the fill tube includes a free end that extends into a mastoidectomy when the electrode array is partially inserted.

4. An ear implant electrode device according to claim 1, wherein the at least a portion of the fill tube is adapted for removal by cutting.

5. An ear implant electrode device according to claim 4, wherein the cutting is flush with the outer surface of the electrode array.

6. An ear implant electrode according to claim 1, wherein the ear implant includes a cochlear implant.

7. An ear implant electrode according to claim 1, wherein the ear implant includes an auditory brainstem (ABI) implant.

8. An ear implant electrode according to claim 1, wherein the ear implant includes a vestibular implant.

9. A method of delivering a therapeutic fluid with an ear implant electrode device, the method comprising:
partially inserting into patient neural tissue an implantable electrode array having:
i. an outer surface including a plurality of electrode contacts for electrically stimulating adjacent neural tissue;
ii. a fluid delivery channel within the electrode array for delivering a therapeutic fluid through at least one fluid delivery port on the outer surface; and
iii. a temporary fill tube connected to the fluid delivery channel;
providing the therapeutic fluid to the fluid delivery channel via the fill tube to deliver the therapeutic fluid through the at least one fluid delivery port to the outer surface of electrode array;
removing at least a portion of the fill tube and completing the insertion of the electrode array into the patient neural tissue so that the fluid delivery channel and any remaining portion of the fill tube are completely enclosed within the neural tissue so as to leave no exposed fluid opening outside the neural tissue and so that the remaining portion of the fill tube is no longer functional.

10. A method according to claim 9, wherein the fill tube includes a free end that extends into a posterior tympanotomy when the electrode array is partially inserted.

11. A method according to claim 9, wherein the fill tube includes a free end that extends into a mastoidectomy when the electrode array is partially inserted.

12. A method according to claim 9, wherein removing the at least a portion of the fill tube includes cutting the fill tube.

13. A method according to claim 12, wherein the cutting is flush with the outer surface of the electrode array.

14. A method according to claim 9, wherein the electrode array includes a cochlear implant electrode array.

15. A method according to claim 9, wherein the electrode array includes an auditory brainstem (ABI) implant electrode array.

16. A method according to claim 9, wherein the electrode array includes a vestibular implant electrode array.
